(19) United States
(12) Patent Application Publication
(10) Pub. No.: US 2020/0060558 A1
(43) Pub. Date: Feb. 27, 2020

(54) PACKAGE-INTEGRATED PIEZOELECTRIC DEVICE FOR BLOOD-PRESSURE MONITORING USING WEARABLE PACKAGE SYSTEMS

(71) Applicant: Intel Corporation, Santa Clara (CA)

(72) Inventors: Aleksandar ALEKSOV, Chandler, AZ (US); Feras EID, Chandler, AZ (US); Sasha N. OSTER, Chandler, AZ (US); Adel ELSHERBINI, Chandler, AZ (US); Johanna M. SWAN, Scottsdale, AZ (US)

(21) Appl. No.: 16/304,070

(22) PCT Filed: Jul. 2, 2016
(86) PCT No.: PCT/US2016/040905

§ 371 Date: Nov. 21, 2018

(51) Int. Cl. A61B 5/021; A61B 5/0408; A61B 5/00; A61B 5/11; H01L 41/08; H01L 41/316; H01L 41/317

(52) U.S. Cl. CPC A61B 5/02141; A61B 5/04085; A61B 5/7278; A61B 5/6833; A61B 5/1102; H01L 41/1876; H01L 41/316; H01L 41/317; A61B 2562/0215; A61B 2562/164; A61B 2562/125; H01L 41/0805

(57) ABSTRACT

Embodiments of the invention include a wearable blood-pressure monitor and methods of forming such devices. In an embodiment, the blood-pressure monitor includes a stretchable substrate. Additionally, a semiconductor die may be embedded within the stretchable substrate. In order to determine blood-pressure, the blood-pressure monitor may include an electrocardiogram sensor and a piezoelectric sensor for detecting a ballistocardiograph response. In an embodiment, both types of sensor may be electrically coupled to the semiconductor die. Embodiments of the invention include a piezoelectric sensor that includes a piezoelectric layer and a first and second electrode. In an embodiment the first electrode is in contact with a first surface of the piezoelectric layer, and the second electrode is in contact with a second surface of the piezoelectric layer that is opposite to the first surface.

### FIELD OF THE INVENTION

Embodiments of the present invention relate generally to the manufacture of wearable systems for monitoring blood-pressure and methods of forming such systems. In particular, embodiments of the present invention relate to the use of piezoelectric sensors for monitoring the heart and methods for manufacturing such devices.

### BACKGROUND OF THE INVENTION

Mobile blood-pressure monitoring (BPM) systems have proven to be a difficult product to successfully manufacture and market. Currently there is no commercially available/viable solution to provide continuous mobile BPM. Instead, currently available BPM systems rely on an inflatable cuff. Such systems require a large battery that is used to periodically inflate and deflate the cuff. Accordingly, this solution is bulky and only allows for measurements at discrete times. Such systems are truly only desirable for people with known blood-pressure problems (high or low) as a healthy person who is not suspected to have any heart/vascular problem would not desire to voluntarily use such a bulky device, especially not on a daily basis or for a prolonged period of time.

However, there are solutions to provide BPM other than inflatable cuffs. Essentially, these solutions require a combination of at least two of the three methods to measure heart rate (i.e., electrocardiography (ECG), ballistocardiography (BCG) and photoplethysmography (PPG)). One such BPM system utilizes a combination of ECG and PPG. There are, however, significant drawbacks to these types of systems as well. Particularly, the use of PPG sensors requires a substantial power budget, because PPG sensors rely on reflection of light from under the skin that is shone by a relatively high power light emitting diode (LED). The power budget of the LED alone can easily surpass the power budget of the rest of the electronic system including low power CPU, ASICs, ECG, motion sensors and Bluetooth Low Energy (BTLE). For example, the power budget of a system (without the LED) may be approximately 45 mW, while a PPG enabled sensor module alone may have a power budget of approximately 50 mW. As such, PPG systems suffer from short battery life or, alternatively, they are bulky due to larger batteries.

Some alternative systems have been proposed that include a combination of BCG and ECG. However these systems suffer from low processing volumes and large form factors (in the X, Y, and Z dimensions). Such systems utilize a discrete piezoelectric patch and attempt to manufacture or assemble the other components (flexible circuit, electrodes, etc.) on top of the discrete piezoelectric patch. Accordingly, the wearability of the system is decreased because the piezoelectric patch limits the flexibility. Additionally, the need to use discrete components increases manufacturing costs and results in relatively large form factors.

Finally, some systems have proposed the combination of PPG and BCG sensors. However, this combination fails to provide a useful device because of the drawbacks from both PPG and BCG. Particularly, the PPG sensor requires a high power budget and the BCG sensor has poor form factor and is not flexible.

### BRIEF DESCRIPTION OF THE DRAWINGS

FIG. 1A is a cross-sectional illustration of a wearable system that includes a piezoelectric sensor embedded below a die, according to an embodiment of the invention.

FIG. 1B is a plan view illustration of a portion of the wearable system that more clearly illustrates the meandering traces, according to an embodiment of the invention.

FIG. 2A is a cross-sectional illustration of a wearable system that includes a piezoelectric sensor that is not formed within the footprint of a die, according to an embodiment of the invention.

FIG. 2B is a cross-sectional illustration of a wearable system that includes a piezoelectric sensor that is formed below a stiffening mass, according to an embodiment of the invention.

FIG. 3A is a cross-sectional illustration of a wearable system that includes a plurality of piezoelectric sensors, according to an embodiment of the invention.

FIG. 3B is a plan view illustration of a portion of the wearable system that more clearly illustrates the meandering traces that electrically couple the plurality of piezoelectric sensors, according to an embodiment of the invention.

FIG. 4A is a cross-sectional illustration of the wearable system after a processing operation for forming the first electrode of the piezoelectric sensor has been performed, according to an embodiment of the invention.

FIG. 4B is a cross-sectional illustration of the wearable system after a processing operation for forming the piezoelectric layer has been performed, according to an embodiment of the invention.

FIG. 4C is a cross-sectional illustration of the wearable system after a processing operation for forming a dielectric layer has been performed, according to an embodiment of the invention.

FIG. 4D is a cross-sectional illustration of the wearable system after a processing operation for forming a seed layer has been performed, according to an embodiment of the invention.

FIG. 4E is a cross-sectional illustration of the wearable system after processing operations for forming and patterning a resist layer have been performed, according to an embodiment of the invention.

FIG. 4F is a cross-sectional illustration of the wearable system after a processing operation for forming the second electrode has been performed, according to an embodiment of the invention.

FIG. 4G is a cross-sectional illustration of the wearable system after processing operations for removing the resist layer and the remaining portions of the seed layer have been performed, according to an embodiment of the invention.

FIG. 4H is a cross-sectional illustration of the wearable system after processing operations for forming subsequent metal layers, integrating a semiconductor die, and forming the stretchable substrate have been performed, according to an embodiment of the invention.

FIG. 4I is a cross-sectional illustration of the wearable system after a processing operation for removing the carrier substrate has been performed, according to an embodiment of the invention.

FIG. 5 is a schematic of a computing device built in accordance with an embodiment of the invention.

### DETAILED DESCRIPTION OF THE INVENTION

Described herein are systems that include piezoelectric sensors for use in mobile BPM and methods of forming such devices. In the following description, various aspects of the illustrative implementations will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative implementations. However, it will be apparent to one skilled in the art that the present invention may be practiced without the specific details. In other instances, well-known features are omitted or simplified in order not to obscure the illustrative implementations.

Various operations will be described as multiple discrete operations, in turn, in a manner that is most helpful in understanding the present invention, however, the order of description should not be construed to imply that these operations are necessarily order dependent. In particular, these operations need not be performed in the order of presentation.

Embodiments of the present invention enable mobile and continuous BPM by combining ECG and BCG signals. In contrast to the discrete BCG sensors described above, embodiments of the present invention utilize BCG sensors that include a piezoelectric element that is built directly into the package by integrating the process flow for creating piezoelectric layers into a wearable/stretchable substrate. Accordingly, embodiments of the invention allow for the fabrication of a BCG module that is highly conformal to the human body and does not significantly increase the form factor of the system. In addition to being more comfortable to wear due to the high level of conformability, flexible packages also provide a more accurate BCG signal since the contact between the skin and the sensor is improved.

Furthermore, embodiments of the invention are able to utilize the power-efficient nature of a piezoelectric sensor to minimize the power budget of the system. Since embodiments of the invention rely on an external mechanical source to compress the piezoelectric layer (i.e., the ballistic response of the heart provides an acoustic wave that compresses the piezoelectric layer), the power budget for the system is minimal. Additionally, in some embodiments, the ECG sensor may only be activated at discrete times (e.g., at regular intervals, or when the BCG sensor detects an anomaly in the heart rate). In such embodiments, selectively turning on the ECG sensor may further reduce the power budget of the system.

Another beneficial aspect of embodiments of the invention is the reduction of fabrication costs. Instead of relying on discrete components that are processed further, embodiments of the present invention may allow for high volume manufacturing. For example, embodiments of the invention include methods for fabricating the piezoelectric layers of the BCG sensor as part of the substrate itself at the panel level (e.g., 0.5 m×0.5 m panels). Accordingly, each panel may contain hundreds of BPM systems that are processed in parallel, thereby significantly reducing the cost of the systems.

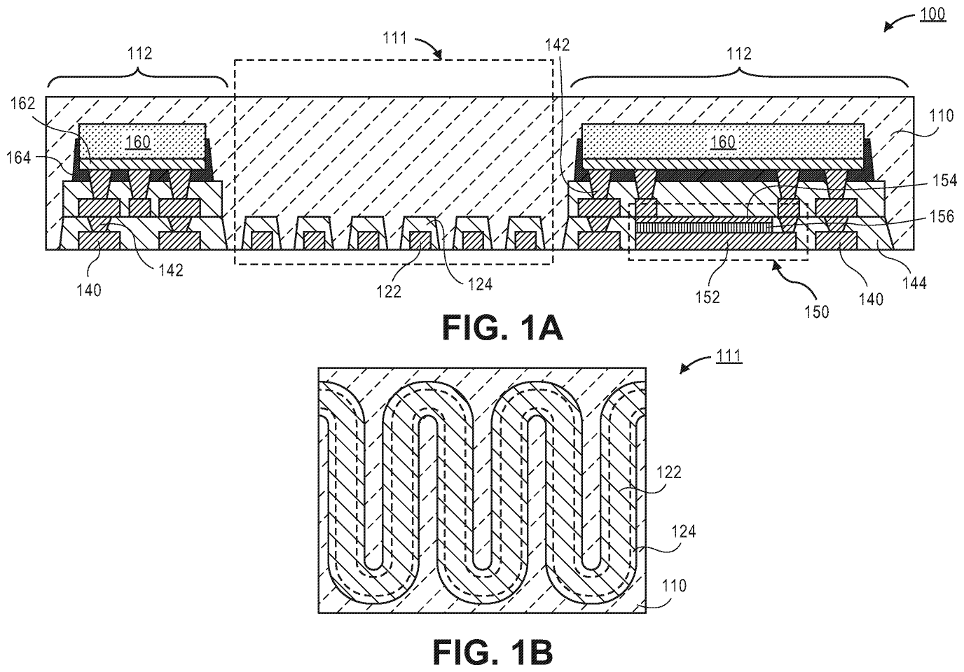

Referring now to FIG. 1A, a cross-sectional illustration of a wearable system 100 is illustrated, according to an embodiment of the invention. As illustrated, the wearable system 100 is formed on a stretchable substrate 110. The stretchable substrate 110 is a compliant material that allows for the wearable system 100 to stretch, flex, bend, twist, etc. The compliant nature of the stretchable substrate 110 may be attributable to a low modulus. For example, the elastic modulus of the stretchable substrate 110 may be less than approximately 10 MPa. In an exemplary embodiment, the elastic modulus of the stretchable substrate 110 may be between approximately 100 kPa and 10 MPa. By way of example, the stretchable substrate 110 may be polydimethylsiloxane (PDMS) or polyurethane.

The wearable system 100 may include one or more stretchable portions 111 and one or more rigid portions 112. According to an embodiment, the stretchable portions 111 include meandering traces 122 that provide electrical connections between components of the wearable system 100, and the rigid portions 112 include one or more semiconductor dice 160. In the illustrated embodiment, the wearable system 100 includes two rigid portions 112. According to an embodiment, each rigid portion 112 may include one or more semiconductor dice 160, however embodiments are not limited to such configurations. For example, more than two semiconductor dice 160 may be included in each rigid portion 112. In an embodiment, the semiconductor die 160 includes one or more electrical devices (not shown). For example, the electrical devices may include one or more of a processor, a memory component, a sensor, a microelectromechanical system (MEMS), or the like, or any combination thereof. In an embodiment the semiconductor die 160 may be a system-on-a-chip (SoC).

According to an embodiment, a modulus of the semiconductor die 160 may be greater than a modulus of the stretchable substrate 110. For example, the elastic modulus of the semiconductor die 160 may be greater than approximately 100 GPa. In an embodiment, the elastic modulus of the semiconductor die 160 may be between approximately 100 GPa and 120 GPa. Accordingly, the greater modulus of the semiconductor die 160 reduces the overall compliance of the rigid portions 112, thereby causing the stretching of the wearable device 100 to be primarily located in the stretchable portions 111.

According to an embodiment, the semiconductor die 160 is a flip-chip semiconductor die. The flip-chip structure of the semiconductor dice 160 may be substantially similar to those presently known in the art. For example, the back end of line interconnect stack 162 of the die 160 may be electrically coupled to vias 142 and pads 140 formed in/on one or more dielectric layers 144. In an embodiment, an underfill material 164 may also be used between the die 160 and the dielectric layers 144. According to an embodiment, the pads 140 and vias 142 may include a conductive stack of materials, such as, but not limited to adhesion promoters, seed layers, copper, silver, gold, or alloys thereof, and oxidation inhibitors. While a flip-chip die 160 is illustrated in FIG. 1A, it is to be appreciated that the semiconductor die 160 is not limited to flip-chip bonding, and other interconnect structures, such as a wire-bonding, are also within the scope of embodiments of the invention.

According to an embodiment, the interconnect lines 122 in the stretchable portion 111 are formed in a meandering pattern in order to allow for the interconnect lines 122 to stretch as the wearable device 100 is stretched. In an embodiment, the interconnect lines 122 may be covered by a dielectric layer 124. The cross-sectional illustration in FIG. 1A illustrates a plurality of portions of a single interconnect line 122. In order to more clearly illustrate the meandering pattern, a plan view of stretchable portion 111 is illustrated in FIG. 1B. As illustrated, the meandering pattern is a repetitive switchback pattern, though other meandering patterns that allow for the interconnect lines 122 to stretch as the wearable device 100 is stretched may also be used. In FIG. 1B, the interconnect line 122 is hidden by the dielectric layer 124, and is therefore represented with dashed lines to indicate that the interconnect line 122 is formed below the dielectric layer 124.

Embodiments of the invention include a meandering pattern that allows for the interconnect lines 122 to stretch approximately 40% or greater without failing (i.e., the length of the fully stretched interconnect lines 124 along the stretched dimension may be approximately 40% longer than the un-stretched length of the interconnect lines 124). Additional embodiments include a meandering pattern that allows for the interconnect lines 124 to stretch between approximately 45% and 55% without failing. The interconnect lines 124 may be any commonly used conductive material for interconnect lines. For example, the interconnect lines 122 may be copper, silver, gold, or alloys thereof. Additional embodiments may further include interconnect lines 122 that are a conductive stack of materials, such as, but not limited to adhesion promoters, seed layers, and oxidation inhibitors.

Referring back to FIG. 1A, the illustrated embodiment includes a piezoelectric sensor 150 that is formed in one of the rigid portions 112. For example, the piezoelectric sensor 150 may be formed substantially within the footprint of a die 160. In the illustrated embodiment, the piezoelectric sensor 150 is integrated into the dielectric layers 144 below the die 160. The piezoelectric sensor 150 may include a piezoelectric layer 156. In an embodiment, a first electrode 152 may be formed in contact with a first surface of the piezoelectric layer 156, and a second electrode 154 may be formed in contact with a second surface of the piezoelectric layer 156 that is opposite the first surface. According to an embodiment, the piezoelectric sensor 150 is able to produce an electrical signal (e.g., a voltage differential) when the piezoelectric layer 156 is compressed. The electrical signal is picked up by the first electrode 152 and the second electrode 154 and delivered to a die 160 by one or more pads 140 and/or vias 142.

In order to provide a usable signal to noise ratio, embodiments of the invention include a high performance piezoelectric material for the piezoelectric layer 156. For example, the high performance piezoelectric layer 156 may be lead zirconate titanate (PZT), potassium sodium niobate (KNN), zinc oxide (ZnO), or combinations thereof. High performance piezoelectric materials such as these typically require a high temperature anneal (e.g., greater than 500° C.) in order to attain the proper crystal structure to provide the piezoelectric effect. As such, currently available piezoelectric actuators require a substrate that is capable of withstanding high temperatures (e.g., silicon). Low melting temperature substrates described herein, such as stretchable substrates and dielectric materials, typically cannot withstand such high temperatures. However, embodiments of the present invention allow for a piezoelectric layer 156 to be formed at much lower temperatures. For example, instead of a high temperature anneal, embodiments include depositing the piezoelectric layer 156 in an amorphous phase and then using a pulsed laser to crystallize the piezoelectric layer 156. For example, the piezoelectric layer 156 may be deposited with a sputtering process, an ink jetting process, or the like. According to an embodiment, the pulsed laser annealing process may use an excimer laser with an energy density between approximately 10-100 mJ/cm² and a pulsewidth between approximately 10-50 nanoseconds. Utilizing such an annealing process allows for the high performance piezoelectric layer 156 to be formed without damaging the materials surrounding the piezoelectric sensor 150.

According to an embodiment, the piezoelectric sensor 150 may be utilized as a BCG sensor to monitor the acoustic waveform produced by the heart of a user. Particularly, as the acoustic waveform from a heartbeat passes the piezoelectric sensor 150, the waveform compresses the piezoelectric layer 156. The compression of the piezoelectric material 156 induces a voltage differential across the piezoelectric layer 156, and the first electrode 152 and the second electrode transfer the electrical signal to the die 160. As such, the piezoelectric sensor 150 may be used to monitor the heartrate of a user wearing the wearable system 100.

As described above, the use of BCG information in conjunction with another heartrate monitoring technique (i.e., ECG or PPG) allow for the blood-pressure to be calculated. In one embodiment, an ECG sensor may also be integrated into the wearable device 100 to provide the electrical waveform to the die 160 in addition to the acoustic waveform detected by the piezoelectric sensor 150. In an embodiment, the ECG sensor may include one or more conductive pads 140 formed on the bottom surface of the stretchable substrate 110. Accordingly, direct contact with the skin may be made and the ECG signal may be sent to a die 160 on the wearable system 100. Additional embodiments may include an ECG signal that is obtained from a sensor that is remote to the wearable system 100. For example, an ECG signal may be wirelessly transmitted (e.g., Bluetooth, Wi-Fi, etc.) to the die 160 in the wearable system 100 by a pacemaker that is external to the wearable system 100. Additional embodiments may also include combing the BCG signal of the piezoelectric sensor 150 with a signal from a PPG sensor (not shown) in order to monitor blood-pressure. Furthermore, some embodiments may include a wearable system 100 that includes a PPG sensor, an ECG sensor, and a piezoelectric BCG sensor.

In an uncalibrated device, the signals obtained from two or more of the piezoelectric sensor 150, and one or both of an ECG sensor and a PPG sensor may be used to monitor changes in the blood-pressure. Alternative embodiments may include a wearable device 100 that can be calibrated. In a calibrated device the signals obtained from two or more of the piezoelectric sensor 150, and one or both of an ECG sensor and a PPG sensor may be used to monitor the actual blood-pressure of a user of the wearable system 100.

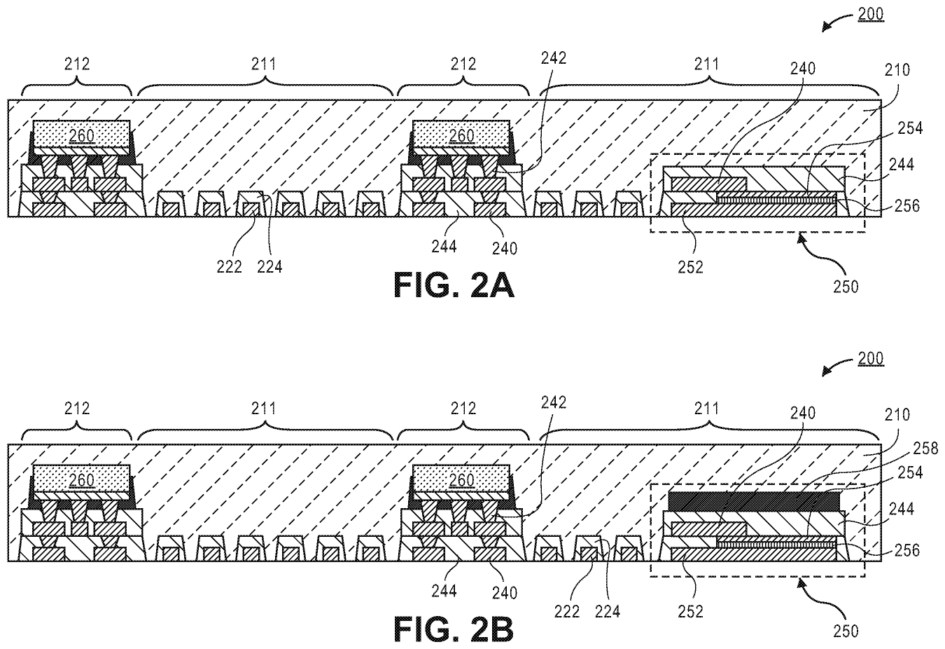

Referring now to FIG. 2A, a cross-sectional illustration of a wearable system 200 with a piezoelectric sensor 250 formed in a stretchable portion 211 is shown, according to an embodiment of the invention. The wearable system 200 may be substantially similar to the wearable system 100, with the exception of the location of the piezoelectric sensor 250. Moving the piezoelectric sensor 250 outside of the rigid portions 212 provides several advantages. One advantage is that the size of the piezoelectric sensor 250 may be increased since the piezoelectric sensor 250 does not need to be within the footprint of a die 260. Increasing the size of the piezoelectric sensor 250 increases the signal strength that may be generated by a heartbeat. Accordingly, the signal to noise ratio may be increased relative to a piezoelectric sensor that has a smaller footprint. The improved signal to noise ratio may provide a more reliable and accurate determination of the heartrate and/or blood-pressure.

Additionally, positioning the piezoelectric sensor 250 in the stretchable portion 211 allows for improved contact with the skin (not shown) of a user of the wearable device 200. While the piezoelectric sensor 250 may not be as stretchable as the meandering traces 222, the piezoelectric sensor 250 may be bendable and/or flexible. As such, the piezoelectric sensor 250 may be able to conform to the surface of the skin better than when the piezoelectric sensor is formed in a rigid portion 212. The improved conformability with the skin of the user allows for improved detection of the heartbeat, thereby providing an increased signal to noise ratio.

Referring now to FIG. 2B, a cross-sectional illustration of wearable device 200 is shown, according to an additional embodiment of the invention. The wearable device 200 in FIG. 2B is substantially similar to the wearable device 200 illustrated in FIG. 2A, and further includes a stiffening mass 258 formed above the piezoelectric sensor 250. Such an embodiment may be utilized when the piezoelectric sensor 250 does not have the necessary stiffness against the skin to allow for an adequate stress to develop inside the piezoelectric layer 256 in order to generate a readable electrical signal. According to an embodiment, the stiffening mass 258 may be a high modulus material and/or a high density material. For example, the stiffening mass 258 may be a metallic material, (e.g., copper, stainless steel, etc.), a ceramic material, or any other material that can provide the requisite stiffness. Additional embodiments may omit the stiffening mass 258 and utilize a thicker second electrode 254. The increased thickness of the second electrode 254 may provide an effect substantially similar to the use of the stiffening mass 258. Furthermore, while a stiffening mass 258 is illustrated as being used in conjunction with the embodiment illustrated in FIG. 2A, it is to be appreciated that a stiffening mass 258 may be used in conjunction with any embodiments described herein in a substantially similar manner.

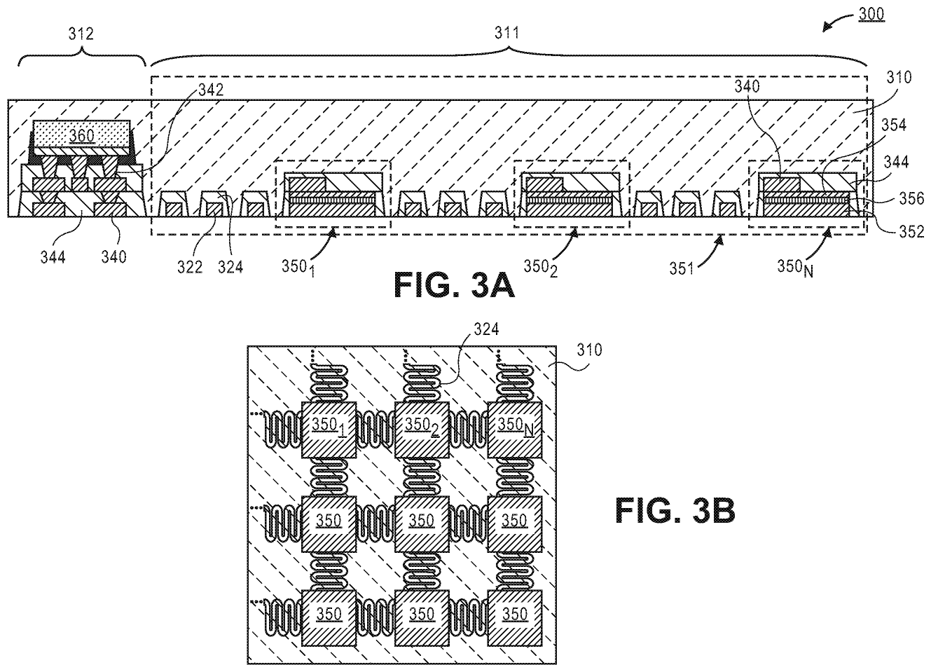

Referring now to FIG. 3A, a cross-sectional illustration of a wearable device 300 is shown, according to an additional embodiment of the invention. The wearable system 300 is substantially similar to the wearable devices 200 described above, with the exception that a plurality of piezoelectric sensors 350₁-350ₙ is arranged in a sensor array 351 in a stretchable portion 311. According to an embodiment, the plurality of piezoelectric sensors 350₁-350ₙ provide a larger total sensing area that may provide an improved signal to noise ratio.

Additionally, embodiments of the invention include electrically coupling the individual piezoelectric sensors 350 with meandering traces 322/324, as illustrated in the plan view shown in FIG. 3B. Accordingly, a high signal to noise ratio may be obtained while still maintaining the ability to stretch. Such an embodiment may, therefore, provide improved contact with a user's skin.

Additionally, embodiments of the invention may include forming the plurality of piezoelectric sensors 350 in a pattern that matches the path of a blood vessel of a user. Such an embodiment may allow for advanced cardio analysis. For example, a plurality of piezoelectric sensors 350₁-350ₙ formed along the length of a blood vessel may allow for the localized distribution of the pressure wave of a user's blood flow to be determined.

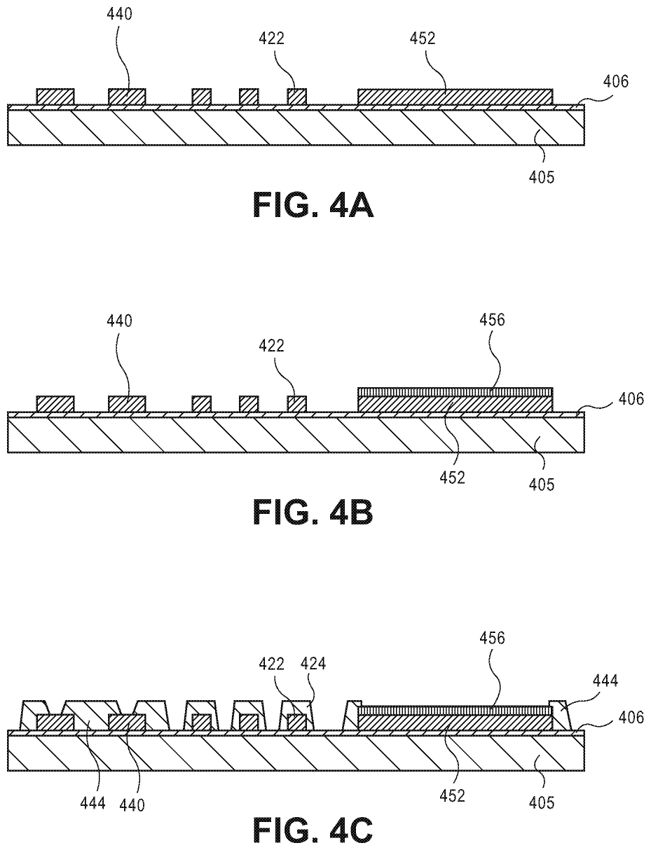

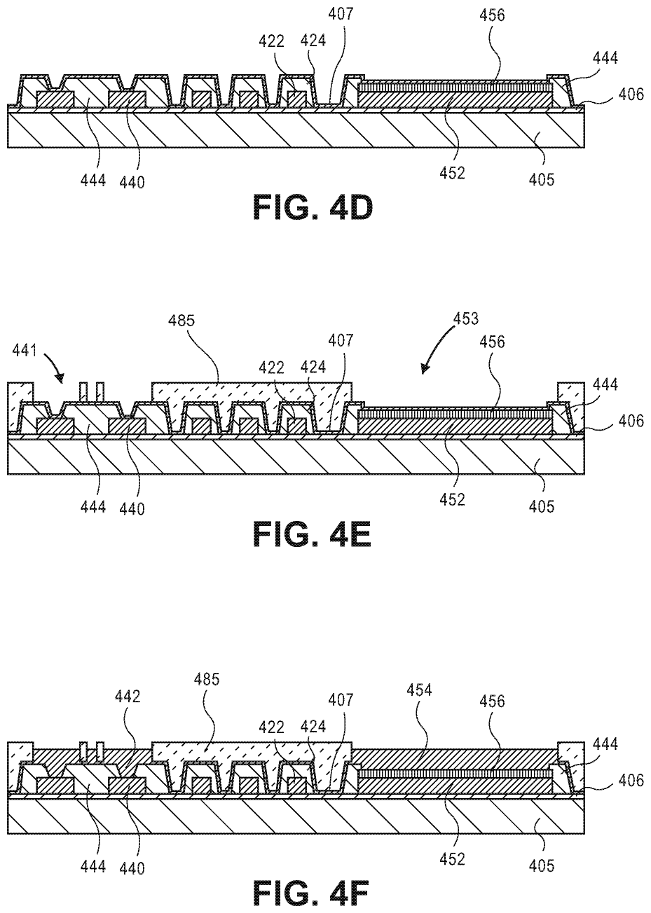

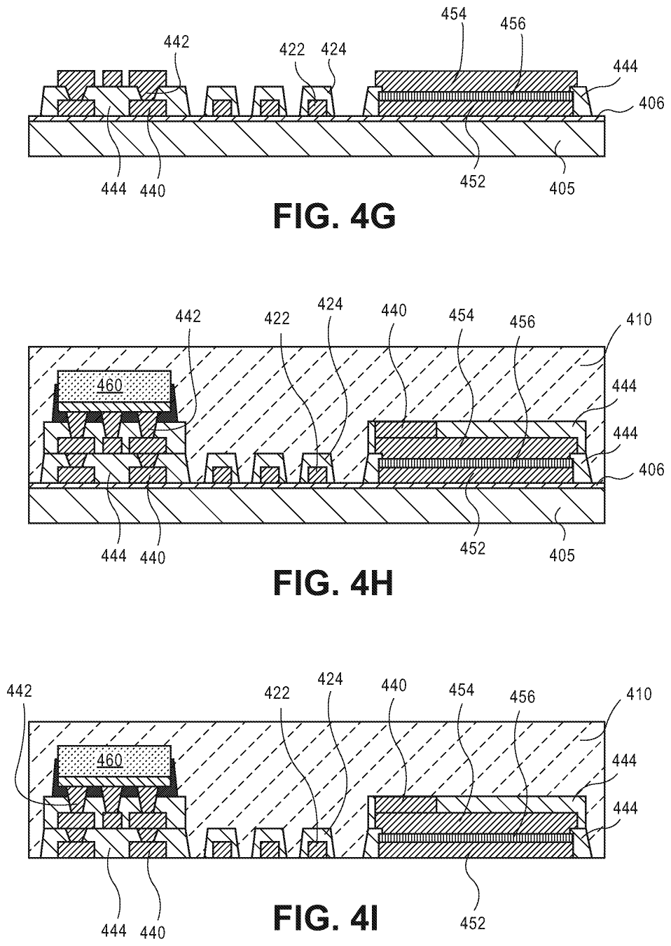

Referring now to FIGS. 4A-4I, a process flow for forming a piezoelectric sensor in a wearable device in accordance with an embodiment of the invention is shown. While a single wearable device is illustrated in FIGS. 4A-4I, it is to be appreciated that a plurality of wearable devices may be fabricated in parallel on the same carrier substrate. For example, a plurality of wearable devices may be fabricated at the panel level (e.g., 0.5 m×0.5 m) or the quarter-panel level. Accordingly, hundreds of wearable devices may be processed in parallel, leading to compatibility with high-volume manufacturing.

Referring now to FIG. 4A, pads 440, meandering traces 422, and the first electrode 452 are formed over a carrier substrate 405. In an embodiment, a release layer 406 may also be formed over the carrier substrate 405 to allow for easier removal of the wearable device in subsequent processing operations.

Referring now to FIG. 4B, the piezoelectric layer 456 may be formed over the first electrode 452. According to an embodiment, the piezoelectric layer may be formed over the first electrode 452 with a selective deposition process, or a patterning process. In order to provide a usable signal to noise ratio, embodiments of the invention include a high performance piezoelectric material for the piezoelectric layer 456. For example, the high performance piezoelectric layer 456 may be PZT, KNN, ZnO, or combinations thereof. High performance piezoelectric materials such as these typically require a high temperature anneal (e.g., greater than 500° C.) in order to attain the proper crystal structure to provide the piezoelectric effect. As such, currently available piezoelectric actuators require a substrate that is capable of withstanding high temperatures (e.g., silicon). Low melting temperature substrates described herein, such as stretchable substrates and dielectric materials, typically cannot withstand such high temperatures. However, embodiments of the present invention allow for a piezoelectric layer 456 to be formed at much lower temperatures. For example, instead of a high temperature anneal, embodiments include depositing the piezoelectric layer 456 in an amorphous phase and then using a pulsed laser to crystallize the piezoelectric layer 456. For example, the piezoelectric layer 456 may be deposited with a sputtering process, an ink jetting process, or the like. According to an embodiment, the pulsed laser annealing process may use an excimer laser with an energy density between approximately 10-100 mJ/cm² and a pulsewidth between approximately 10-50 nanoseconds. Utilizing such an annealing process allows for the high performance piezoelectric layer 456 to be formed without damaging the surrounding layers on which the piezoelectric sensor 450 is formed.

Referring now to FIG. 4C, a dielectric layer is deposited and patterned to form the dielectric covering 424 over the meandering traces 422 and the dielectric layer 444 over pads 440 and the first electrode 452. In an embodiment, the dielectric layer may be patterned to provide openings for subsequent conductive layers. The deposition and patterning of the dielectric layer may include standard electronics packaging operations (e.g., blanket deposition of a photo-imagable dielectric, followed by exposure, developing, and curing of the photo-imagable dielectric).

Referring now to FIG. 4D, a seed layer 407 may be formed over the exposed surfaces. The formation of the seed layer 407 allows for subsequent plating of conductive features and may be performed with techniques and processes known to those with skill in the electronics packaging arts. Thereafter, lithography for a second metal layer may be implemented, as illustrated in FIG. 4E. For example, the lithography may include standard processes and materials, such as the deposition of a dry-film resist 485, exposure of the dry-film resist 485, and developing of the dry-film resist 485 to form openings 441 and 453 for forming the subsequent metal layer.

Referring now to FIG. 4F, the subsequent metal layer is formed. For example, the exposed portions of the seed layer 407 may be used to selectively deposit conductive material with a plating process, such as electroplating. As illustrated, the plating process may be used to form vias 442, pads 440, and the second electrode 454.

Subsequent to the plating process in FIG. 4F, embodiments of the invention may include stripping the dry-film resist 485 and removing the remaining portions of the seed layer 407, as illustrated in FIG. 4G. For example, the dry-film resist 485 may be removed with an ashing process and the seed layer 407 may be removed with an etching process.

Referring now to FIG. 4H, embodiments of the invention may include subsequent processing operations to form any additional dielectric layers 444, conductive layers (e.g., conductive pads 440, vias 442, traces 422, etc.), and integration of needed dice 460.

Additionally, a stretchable substrate 410 may be deposited over the electrical components. In an embodiment, the stretchable substrate 410 may be formed with a pouring process. For example, a precursor liquid may be poured over the carrier 405 and then polymerized and cross-linked to form the stretchable substrate 410.

Referring now to FIG. 4I, the carrier 405 and the release layer 406 may be removed after the formation of stretchable substrate 410 is completed. By way of example, the carrier 405 may be removed with a delamination process, an etching process, or any other processes known in the art. Additionally, when a plurality of wearable systems are formed in parallel, a dicing or other singulation operation may be implemented to separate each device, in accordance with embodiments of the invention.

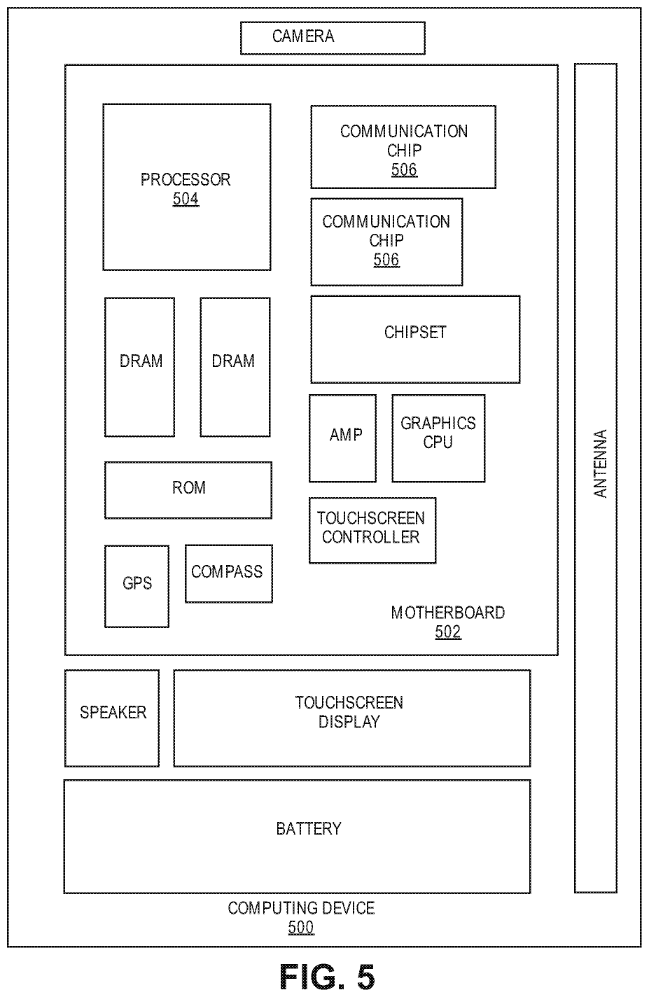

FIG. 5 illustrates a computing device 500 in accordance with one implementation of the invention. The computing device 500 houses a board 502. The board 502 may include a number of components, including but not limited to a processor 504 and at least one communication chip 506. The processor 504 is physically and electrically coupled to the board 502. In some implementations the at least one communication chip 506 is also physically and electrically coupled to the board 502. In further implementations, the communication chip 506 is part of the processor 504.

Depending on its applications, computing device 500 may include other components that may or may not be physically and electrically coupled to the board 502. These other components include, but are not limited to, volatile memory (e.g., DRAM), non-volatile memory (e.g., ROM), flash memory, a graphics processor, a digital signal processor, a crypto processor, a chipset, an antenna, a display, a touchscreen display, a touchscreen controller, a battery, an audio codec, a video codec, a power amplifier, a global positioning system (GPS) device, a compass, an accelerometer, a gyroscope, a speaker, a camera, and a mass storage device (such as hard disk drive, compact disk (CD), digital versatile disk (DVD), and so forth).

The communication chip 506 enables wireless communications for the transfer of data to and from the computing device 500. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. The communication chip 506 may implement any of a number of wireless standards or protocols, including but not limited to Wi-Fi (IEEE 802.11 family), WiMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, derivatives thereof, as well as any other wireless protocols that are designated as 3G, 4G, 5G, and beyond. The computing device 500 may include a plurality of communication chips 506. For instance, a first communication chip 506 may be dedicated to shorter range wireless communications such as Wi-Fi and Bluetooth and a second communication chip 506 may be dedicated to longer range wireless communications such as GPS, EDGE, GPRS, CDMA, WiMAX, LTE, Ev-DO, and others.

The processor 504 of the computing device 500 includes an integrated circuit die packaged within the processor 504. In some implementations of the invention, the integrated circuit die of the processor may be packaged on a stretchable substrate that includes a piezoelectric sensor for determining heartrate, in accordance with implementations of the invention. The term “processor” may refer to any device or portion of a device that processes electronic data from registers and/or memory to transform that electronic data into other electronic data that may be stored in registers and/or memory.

The communication chip 506 also includes an integrated circuit die packaged within the communication chip 506. In accordance with another implementation of the invention, the integrated circuit die of the communication chip may be packaged on a stretchable substrate that includes a piezoelectric sensor for determining heartrate, in accordance with implementations of the invention.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize.

These modifications may be made to the invention in light of the above detailed description. The terms used in the following claims should not be construed to limit the invention to the specific implementations disclosed in the specification and the claims. Rather, the scope of the invention is to be determined entirely by the following claims, which are to be construed in accordance with established doctrines of claim interpretation.

Embodiments of the invention include a wearable device, comprising: a piezoelectric sensor embedded within a stretchable substrate, wherein the piezoelectric sensor comprises: a piezoelectric layer; a first electrode formed in contact with a first surface of the piezoelectric layer; and a second electrode formed in contact with a second surface of the piezoelectric layer that is opposite to the first surface.

Additional embodiments of the invention include a wearable device, wherein the wearable device comprises a rigid portion and a stretchable portion.

Additional embodiments of the invention include a wearable device, wherein the rigid portion comprises a semiconductor die, and wherein the stretchable portion comprises meandering traces.

Additional embodiments of the invention include a wearable device, wherein the piezoelectric sensor is formed in the rigid portion, and wherein the piezoelectric sensor is formed within the footprint of the semiconductor die.

Additional embodiments of the invention include a wearable device, wherein the piezoelectric sensor is formed in the stretchable portion.

Additional embodiments of the invention include a wearable device, wherein the piezoelectric sensor is electrically coupled to the semiconductor die by one or more meandering traces.

Additional embodiments of the invention include a wearable device, further comprising a stiffening mass formed above the piezoelectric sensor.

Additional embodiments of the invention include a wearable device, further comprising a plurality of piezoelectric sensors formed in the stretchable portion.

Additional embodiments of the invention include a wearable device, wherein the plurality of piezoelectric sensors are electrically coupled to each other by meandering traces.

Additional embodiments of the invention include a wearable device, wherein the plurality of piezoelectric sensors are arranged in a pattern similar to the shape of a blood vessel.

Additional embodiments of the invention include a wearable device, further comprising one or more stiffening masses formed above the plurality of piezoelectric sensors.

Additional embodiments of the invention include a wearable device, further comprising an electrocardiogram sensor.

Additional embodiments of the invention include a wearable device, wherein the semiconductor die is configured to receive signals from the electrocardiogram sensor and the piezoelectric sensor and generate a blood-pressure measurement.

Additional embodiments of the invention include a wearable device, wherein the blood-pressure measurement is an absolute blood-pressure measurement.

Embodiments of the invention include a method of forming a piezoelectric sensor in a wearable device, comprising: forming a first electrode on a carrier substrate; forming a piezoelectric layer on the first electrode; forming a second electrode over the piezoelectric layer; forming a stretchable substrate over the carrier substrate, wherein the stretchable substrate encases the first electrode, the piezoelectric layer, and the second electrode; and removing the stretchable substrate from the carrier substrate.

Additional embodiments of the invention include a method of forming a piezoelectric sensor in a wearable device, wherein forming the piezoelectric layer, comprises: depositing the piezoelectric layer over the first electrode, wherein the piezo-electric layer is an amorphous layer; and crystallizing the piezoelectric layer with a pulsed laser anneal.

Additional embodiments of the invention include a method of forming a piezoelectric sensor in a wearable device, wherein the piezoelectric layer is deposited with a sputtering or ink-jetting process.

Additional embodiments of the invention include a method of forming a piezoelectric sensor in a wearable device, wherein the pulsed laser anneal is performed with an Excimer laser with an energy density in the range of approximately 10-100 mJ/cm² and pulse width in the range of approximately 10-50 nanoseconds.

Additional embodiments of the invention include a method of forming a piezoelectric sensor in a wearable device, wherein the piezoelectric layer is lead zirconate titanate (PZT), potassium sodium niobate (KNN), or zinc oxide (ZnO).

Additional embodiments of the invention include a method of forming a piezoelectric sensor in a wearable device, wherein a plurality of wearable devices are formed on a single carrier substrate.

Embodiments of the invention include a wearable blood-pressure monitor, comprising: a stretchable substrate; a semiconductor die embedded in the stretchable substrate; an electrocardiogram sensor electrically coupled to the semiconductor die; and a ballistocardiograph sensor embedded within the stretchable substrate and electrically coupled to the semiconductor die, wherein the ballistocardiograph sensor is a piezoelectric sensor that comprises: a piezoelectric layer; a first electrode formed in contact with a first surface of the piezoelectric layer; and a second electrode formed in contact with a second surface of the piezoelectric layer that is opposite to the first surface.

Additional embodiments of the invention include a wearable blood-pressure monitor, wherein the semiconductor die is configured to receive signals from the electrocardiogram sensor and the ballistocardiograph sensor and generate a blood-pressure measurement.

Additional embodiments of the invention include a wearable blood-pressure monitor, wherein the piezoelectric sensor is formed within the footprint of the semiconductor die.

Additional embodiments of the invention include a wearable blood-pressure monitor, wherein the piezoelectric sensor is not formed within the footprint of the semiconductor die, and wherein the piezoelectric sensor is coupled to the semiconductor die by one or more meandering traces.

Additional embodiments of the invention include a wearable blood-pressure monitor, further comprising a plurality of piezoelectric sensors, wherein the plurality of piezoelectric sensors are electrically coupled to each other by meandering traces, and wherein the plurality of piezoelectric sensors are arranged in a pattern similar to the shape of a blood vessel

What is claimed is:

1. A wearable device, comprising:
a piezoelectric sensor embedded within a stretchable substrate, wherein the piezoelectric sensor comprises:
a piezoelectric layer;
a first electrode formed in contact with a first surface of the piezoelectric layer; and
a second electrode formed in contact with a second surface of the piezoelectric layer that is opposite to the first surface.

2. The wearable device of claim 1, wherein the wearable device comprises a rigid portion and a stretchable portion.

3. The wearable device of claim 2, wherein the rigid portion comprises a semiconductor die, and wherein the stretchable portion comprises meandering traces.

4. The wearable device of claim 3, wherein the piezoelectric sensor is formed in the rigid portion, and wherein the piezoelectric sensor is formed within the footprint of the semiconductor die.

5. The wearable device of claim 3, wherein the piezoelectric sensor is formed in the stretchable portion.

6. The wearable device of claim 5, wherein the piezoelectric sensor is electrically coupled to the semiconductor die by one or more meandering traces.

7. The wearable device of claim 5, further comprising a stiffening mass formed above the piezoelectric sensor.

8. The wearable device of claim 5, further comprising a plurality of piezoelectric sensors formed in the stretchable portion.

9. The wearable device of claim 8, wherein the plurality of piezoelectric sensors are electrically coupled to each other by meandering traces.

10. The wearable device of claim 8, wherein the plurality of piezoelectric sensors are arranged in a pattern similar to the shape of a blood vessel.

11. The wearable device of claim 8, further comprising one or more stiffening masses formed above the plurality of piezoelectric sensors.

12. The wearable device of claim 1, further comprising an electrocardiogram sensor.

13. The wearable device of claim 12, wherein the semiconductor die is configured to receive signals from the electrocardiogram sensor and the piezoelectric sensor and generate a blood-pressure measurement.

14. The wearable device of claim 13, wherein the blood-pressure measurement is an absolute blood-pressure measurement.

15. A method of forming a piezoelectric sensor in a wearable device, comprising:
forming a first electrode on a carrier substrate;
forming a piezoelectric layer on the first electrode;
forming a second electrode over the piezoelectric layer;
forming a stretchable substrate over the carrier substrate, wherein the stretchable substrate encases the first electrode, the piezoelectric layer, and the second electrode; and
removing the stretchable substrate from the carrier substrate.

16. The method of claim 15, wherein forming the piezoelectric layer, comprises:
depositing the piezoelectric layer over the first electrode, wherein the piezo-electric layer is an amorphous layer; and
crystallizing the piezoelectric layer with a pulsed laser anneal.

17. The method of claim 16, wherein the piezoelectric layer is deposited with a sputtering or ink-jetting process.

18. The method of claim 16, wherein the pulsed laser anneal is performed with an Excimer laser with an energy density in the range of approximately 10-100 mJ/cm² and pulse width in the range of approximately 10-50 nanoseconds.

19. The method of claim 15, wherein the piezoelectric layer is lead zirconate titanate (PZT), potassium sodium niobate (KNN), or zinc oxide (ZnO).

20. The method of claim 15, wherein a plurality of wearable devices are formed on a single carrier substrate.

21. A wearable blood-pressure monitor, comprising:
a stretchable substrate;
a semiconductor die embedded in the stretchable substrate;
an electrocardiogram sensor electrically coupled to the semiconductor die; and
a ballistocardiograph sensor embedded within the stretchable substrate and electrically coupled to the semiconductor die, wherein the ballistocardiograph sensor is a piezoelectric sensor that comprises:
a piezoelectric layer;
a first electrode formed in contact with a first surface of the piezoelectric layer; and
a second electrode formed in contact with a second surface of the piezoelectric layer that is opposite to the first surface.

22. The wearable blood-pressure monitor of claim 21, wherein the semiconductor die is configured to receive signals from the electrocardiogram sensor and the ballistocardiograph sensor and generate a blood-pressure measurement.

23. The wearable blood-pressure monitor of claim 21, wherein the piezoelectric sensor is formed within the footprint of the semiconductor die.

24. The wearable blood-pressure monitor of claim 21, wherein the piezoelectric sensor is not formed within the footprint of the semiconductor die, and wherein the piezoelectric sensor is coupled to the semiconductor die by one or more meandering traces.

25. The wearable blood-pressure monitor of claim 24, further comprising a plurality of piezoelectric sensors, wherein the plurality of piezoelectric sensors are electrically coupled to each other by meandering traces, and wherein the plurality of piezoelectric sensors are arranged in a pattern similar to the shape of a blood vessel.
